(19) United States
(12) Patent Application Publication
(10) Pub. No.: US 2018/0263869 A1
(43) Pub. Date: Sep. 20, 2018

(54) PHOTOSENSITIVE CAPSULES, SUNSCREEN COMPOSITIONS COMPRISING THE CAPSULES, AND METHODS OF USE

(71) Applicants: Susan HALPERN CHIRCH, Basking Ridge, NJ (US); Chunhua Li, Hillsborough, NJ (US); Miao Wang, Westfield, NJ (US)

(72) Inventors: Susan HALPERN CHIRCH, Basking Ridge, NJ (US); Chunhua Li, Hillsborough, NJ (US); Miao Wang, Westfield, NJ (US)

(21) Appl. No.: 15/532,617

(22) PCT Filed: Dec. 4, 2015
(86) PCT No.: PCT/IB2015/002519

§ 371 Date: Jun. 2, 2017

Related U.S. Application Data
(60) Provisional application No. 62/087,859, filed on Dec. 5, 2014.

(51) Int. Cl. A61K 8/11; A61K 8/04; A61Q 19/10; A61K 8/40

(52) U.S. Cl. CPC A61K 8/11; A61K 8/40; A61Q 19/10; A61K 8/042

(57) ABSTRACT

The instant disclosure relates to photosensitive capsules (sometimes referred to as “smart capsules”) and sunscreen compositions comprising the capsules that automatically adjust the Sun Protection Factor (“SPF”) of the sunscreen composition during use to provide protection against ultraviolet (“UV”) light, as needed, based on actual exposure to UV light. The photosensitive capsules comprise: (i) a polymeric photosensitive degradable shell; and (ii) one or more UV filters encapsulated by the photosensitive degradable shell, wherein the one or more UV filters is released from the photosensitive degradable shell upon exposure to UV light.

### FIELD OF THE DISCLOSURE

The instant disclosure relates to photosensitive capsules (sometimes referred to as “smart capsules”) and sunscreen compositions comprising the capsules that automatically adjust the Sun Protection Factor (“SPF”) of the sunscreen composition during use to provide protection against ultraviolet (“UV”) light, as needed, based on actual exposure to UV light.

### BACKGROUND

The negative effects of exposure to ultraviolet light are well-known. Prolonged exposure to sunlight causes damage such as sunburn to the skin and dries out hair making it brittle. When skin is exposed to UV light having a wavelength of from about 290 nm to about 400 nm, long term damage can lead to serious conditions such as skin cancer.

UV light also contributes to aging by causing free radicals to form in the skin. Free radicals include, for example, singlet oxygen, hydroxyl radical, the superoxide anion, nitric oxide and hydrogen radicals. Free radicals attack DNA, membrane lipids and proteins, generating carbon radicals. These in turn react with oxygen to produce a peroxyl radical that can attack adjacent fatty acids to generate new carbon radicals. This cascade leads to a chain reaction producing lipid peroxidation products. Damage to the cell membrane results in loss of cell permeability, increased intercellular ionic concentration, and decreased ability to excrete or detoxify waste products. The end result is a loss of skin elasticity and the appearance of wrinkles. This process is commonly referred to as photo-aging.

Concerns exist that certain ingredients sometimes included in sunscreen composition may protect again the damaging effects of UV light but nonetheless pose other health risks to the consumer. For example, 4-Aminobenzoic acid (PABA) has been reported to cause DNA damage, and therefore was banned as a sunscreen ingredient. Therefore, there is a need to protect skin from the damaging effects of UV in a safe and effective manner.

### SUMMARY OF THE INVENTION

The instant disclosure relates to photosensitive capsules (sometimes referred to as “smart capsules”) that encapsulate one or more UV filters and are incorporated into sunscreen compositions. The capsules are typically nanocapsules or microcapsules. The photosensitive capsules are unique in that they automatically adjust the Sun Protection Factor (“SPF”) of the sunscreen composition during use when exposed to UV light. The capsules typically include: (a) a polymeric photosensitive degradable shell; and (b) one or more UV filters encapsulated by the photosensitive degradable shell. Upon exposure to UV light, the polymeric photosensitive degradable shell breaks-apart and releases the one or more UV filters so that the UV filters are available to protect against the damaging effects of UV light.

Sunscreen compositions comprising the photosensitive capsules provide a variety of benefits. For example, by encapsulating the one or more UV filters in a photosensitive degradable shell, the UV filter are substantially isolated from other components of the composition, other components that may negatively interfere or interact with the UV filters. This simplifies formulations and improves shelf-life. Likewise, UV filters that negatively influence each other (and are therefore not typically used together) can be separately encapsulated into different photosensitive capsules, and the different photosensitive capsules can be used together in a single sunscreen composition. Furthermore, many UV filters tend to be oily and impart a “greasy” or other undesirable sensation to the consumer. By encapsulating the one or more UV filters, the aesthetics and “feel” of the sunscreen composition can be improved (e.g., the “greasy” sensation can be minimized or eliminated).

In use, the photosensitive capsules keep the UV filters from coming into direct contact with a consumer's skin unless the skin becomes exposed to UV light, in which case the photosensitive capsules immediately go to work to protect the skin from the UV light. Upon exposure to UV light, the photosensitive degradable shell automatically breaks apart (self-destructs) and releases the UV filters. Once released, the UV filters are fully available to protect the skin by absorbing and/or blocking the UV light.

The photosensitive capsules of the instant disclosure typically include: (i) a polymeric photosensitive degradable shell; and (ii) one or more UV filters encapsulated by the photosensitive degradable shell, wherein the one or more UV filters is released from the photosensitive degradable shell upon exposure to UV light. The photosensitive degradable shell is “photosensitive” because it contains a polymer having photo-responsive groups that react in response to UV light to degrade the polymer, thereby releasing the one or more UV filters from the microcapsule. The polymer may be, for example, a polyacrylate, a polyurea, a polyurethane, polyester, cellulose, starch, a polysaccharide, a silicone, etc. The photo-responsive groups may be, for example, an azobenzene group, a nitrobenzyl group, a quinone-methide group, an o-nitrobenzyl group, etc. In some instance, the photo-responsive group is a 6-nitro-veratroyloxycarbonyl groups containing two methoxy groups on the benzene ring.

The one or more UV filters that may be encapsulated by the polymeric photosensitive degradable shell can be any UV filter known in the art. A single UV filter may be used or a combination of UV filters may be used. For example, the UV filter may be one or more organic UV filters and/or one or more inorganic UV filters. In some cases, the UV filter is drometrizole trisiloxane (Mexoryl XL).

The size of the capsules may vary. The capsules may be nanocapsules or microcapsules. In some cases, the capsules have an average diameter size of about 50 nm to about 850 μm. Typically, 50% or more of the one or more UV filters is released from the photosensitive degradable shell (released from the capsules) within 30 minutes upon exposure to UV light having a wavelength of 350 nm.

In addition to one or more UV filters, other components of sunscreen compositions can be encapsulated by the photosensitive degradable shell, especially components that may difficult to formulate into the sunscreen composition or components that are preferably not continuously in contact with the skin or body of a consumer. The other components that can be encapsulated may be encapsulated together with the one or more UV filters, or separately encapsulated into separate photosensitive capsules. Non-limiting examples of other components that may be encapsulated include SPF boosters, pigments, wetting agents, surfactants, stabilizers, other active ingredients, compounds that exhibit a cooling sensation (or other sensation) on the skin, etc.

In some cases, the capsules include one or more compounds that impart a cooling sensation to the skin. For example, the one or more compounds that impart a cooling sensation to the skin may be substituted cyclohexanols and their esters, carboxamides, menthone ketals, menthoxypropanediols, etc. Non-limiting examples of compounds that impart a cooling sensation to the skin include menthol, isopulegol, menthyl lactate, N-ethyl-3-p-menthane carboxamide (WS-3), 2-isopropyl-N,2,3-trimethyl butanamide (WS-23), N-ethoxycarbonylmethyl-3-p-menthane carboxamide (WS-5), monomenthyl glutarate, monomenthyl succinate, and mixtures thereof.

The photosensitive capsules, in some instances, include (i) about 10 wt. % to about 60 wt. % of a polymeric photosensitive degradable shell; and (ii) about 10 wt. % to about 75 wt. % of one or more UV filters encapsulated by the photosensitive degradable shell, wherein the percent by weight is based on the total weight of the particle. Nonetheless, the amount of polymer photosensitive degradable shell and the amount of the one or more UV filters can vary greatly depending on the material used to generate the shell, the type(s) of UV filters employed, and the desired speed at which the particles are design to release the one or more UV filters.

In one embodiment, the instant disclosure is directed to a sunscreen composition comprising the capsuled described herein. Typically, the sunscreen composition will include both: the photosensitive capsules disclosed herein; and a cosmetically acceptable carrier. The cosmetically acceptable carrier may be aqueous, non-aqueous, an alcohol, or an oil. In some cases, the sunscreen composition is an emulsion, and therefore will typically include one or more emulsifiers (for example, gum Arabic).

In some embodiments, the sunscreen composition includes one or more film-formers (e.g., a copolymer of vinyl acetate and vinyl pyrrolidone), a booster, a pigment, a surfactant, a perfume, etc. Non-limiting example boosters include styrene/acrylates copolymer, calcium aluminum borosilicate, sodium borosilicate particulates, calcium/sodium borosilicate hollow microspheres, and calcium/sodium borosilicate microspheres.

The amount of photosensitive capsules used in a particular sunscreen composition can vary depending on the desired properties of the final product. For example, the sunscreen composition can include: (a) about 5 wt. % to about 50 wt. % of the photosensitive capsules; and (b) a cosmetically acceptable carrier. Although the capsules typically include one or more UV filters, in some instances it may be desirable to have one or more UV filters in the sunscreen composition that are not encapsulated by the photosensitive capsules. The sunscreen composition may include a combination of both capsules comprising one or more UV filters and additionally one or more UV filters that are not part of the capsules. In other instances, it may be desirable to exclude UV filters from the capsules and instead include other components in the capsules (e.g., boosters, compounds that impart a cooling sensation, etc.).

In one embodiment, a sunscreen composition comprises; (a) about 5 wt. % to about 50 wt. %, based on the total weight of the sunscreen composition, of photosensitive capsules having an average diameter size of about 50 nm to about 850 μm, wherein the capsules comprise; (i) about 10 wt. % to about 60 wt. %, based on the total weight of the capsules, of a polymeric photosensitive degradable shell comprising a polymer having photo-responsive groups that react in response to UV light to degrade the polymer, wherein the polymer is selected from the group consisting of a polyacrylate, a polyurea, a polyurethane, polyester, cellulose, starch, a polysaccharide, and a silicone, and wherein the photo-responsive groups are selected from the group consisting of an azobenzene group, a nitrobenzyl group, a quinone-methide group, and an o-nitrobenzyl group; and (ii) about 10 wt. % to about 75 wt. %, based on the total weight of the capsules, of one or more UV filters encapsulated by the photosensitive degradable shell; and (b) a cosmetically acceptable carrier.

In some cases, the sunscreen composition may be transparent, pearlescent, or colored. Furthermore, the sunscreen composition may be in the form of a spray-on product, a lotion, a cream, a gel, a lipstick, a foundation, a moisturizer, a powder, etc.

The instant disclosure also relates to methods of using the photosensitive capsules and sunscreen compositions described herein. For example, the photosensitive capsules and sunscreen composition may be used in a method for protecting a keratinous substrate from UV radiation comprising applying a sunscreen composition of claim 7 to the keratinous substrate. In some cases, the keratinous substrate is skin or hair.

### DETAILED DESCRIPTION

Where the following terms are used in this specification, they are used as defined below.

The terms “comprising,” “having,” and “including” are used in their open, non-limiting sense.

The terms “a” and “the” are understood to encompass the plural as well as the singular.

As used herein, the expression “at least one” means one or more and thus includes individual components as well as mixtures/combinations.

“Cosmetically acceptable” means that the item in question is compatible with any keratinous substrate. For example, “cosmetically acceptable carrier” means a carrier that is compatible with any keratinous substrate. A “physiologically acceptable medium” means a medium which is not toxic and can be applied to the skin, lips, hair, scalp, lashes, brows, nails or any other cutaneous region of the body. Unless otherwise specified, a “cosmetically acceptable carrier” is a “physiologically acceptable medium,” i.e., it is not toxic and can be applied to the skin, lips, hair, scalp, lashes, brows, nails or any other cutaneous region of the body.

The phrase “essentially without” or “essentially free of” refers to less than or equal to 0.5, 0.1, 0.05 or 0.01 wt. %.

The phrase “stable emulsion” refers to a composition that does not undergo phase separation up to a temperature of 45 C°.

The term “SPF” refers to “sun protection factor.”

The term “UV” refers to ultraviolet.

The term “clear” or “transparent” refers to the clarity of a formulation measured by the transmittance percentage of light with a wavelength of 700 nm by UV-Visible spectrophotometry. “Clear” or “transparent” compositions, as used herein, are composition allowing for 90% and 100% of the light to pass through the composition.

The photosensitive capsules of the instant disclosure typically include: (i) a polymeric photosensitive degradable shell; and (ii) one or more UV filters encapsulated by the photosensitive degradable shell, wherein the one or more UV filters is released from the photosensitive degradable shell upon exposure to UV light. The one or more UV filters that may be encapsulated by the polymeric photosensitive degradable shell can be any UV filter known in the art. A single UV filter may be used or a combination of UV filters may be used. The size of the capsules may vary. The capsules may be nanocapsules or microcapsules. In some cases, the capsules have an average diameter size of about 50 nm to about 850 μm. In other embodiments, the average diameter size of the capsules is about 50 nm to about 500 nm, or about 100 nm to about 300 nm. In some cases, the size of the capsules may be from about 1 μm to about 850 μm, from about 5 μm to about 600 μm, from about 10 μm to about 500 μm, or from about 50 μm to about 500 μm. The capsules may be formulated so that the size falls within ranges derived from any of the end-points described herein.

Typically, 50% or more of the one or more UV filters is released from the photosensitive degradable shell (released from the capsules) within 30 minutes upon exposure to UV light having a wavelength of 350 nm. In some cases, it may be desirable to have more (or less) than 50% of the one or more UV filters release within a short or longer period of time. For example, in some cases, 25%, 30%, 45%, 50%, 60%, 70%, 75%, 80%, 90% or more of the one or more UV filters is releases from the capsules within about 1, 2, 3, 4, 5, 6, 7, 8, 9, 10, 20, 30, 40, 45, or 50 minutes, or 1, 2, 3, 4, or 5 hours (or more) after exposure to UV-A light and/or UV-B light (or other types of light). UV-A light typically has a wavelength of 315 nm to 400 nm. UV-B light typically has a wavelength of 280 nm to 315 nm. In some cases, the photosensitive degradable shell degrades upon irradiation with ultraviolet light having a wavelength of about 100 nm to about 500 nm. In some cases the wavelength may be from about 200 nm to about 400 nm. In some embodiments, it may be desirable for the capsules to release their content upon exposure to visible light, i.e., light having a wavelength of about 380 nm to about 750 nm).

The photosensitive capsules, in some instances, include (i) about 10 wt. % to about 60 wt. % of a polymeric photosensitive degradable shell; and (ii) about 10 wt. % to about 75 wt. % of one or more UV filters encapsulated by the photosensitive degradable shell, wherein the percent by weight is based on the total weight of the particle. These amounts, however, can vary greatly depending on the desired properties for the capsules (and the product in which the capsules are incorporated. For example, in some instances, the photosensitive capsules include (i) about 5, 10, 15, 20, or 25 wt. % to about 30, 40, 50, 60, 70, 75, or 80 wt. % of a polymeric photosensitive degradable shell, based on the total weight of the capsules. The photosensitive degradable shell may consist of only the polymer(s) having the photo-responsive groups or may include other components. The amount of the one or more UV filters incorporated into the photosensitive capsules may vary greatly. The particles may comprises, for example, about 5, 10, 15, 20, 25, 30, 35, 40, 45, 50, 55, 60, 65, 70, 75, 80, or 80 wt. %, based on the total weight of the particles, of the one or more UV filters (or the particles can be formulated to include a range of UV filters derived from any of the values set forth above).

In addition to one or more UV filters, other components of sunscreen compositions can be encapsulated by the photosensitive degradable shell, especially components that may difficult to formulate into the sunscreen composition or components that are preferably not continuously in contact with the skin or body of a consumer. The other components that can be encapsulated may be encapsulated together with the one or more UV filters, or separately encapsulated into separate photosensitive capsules. Non-limiting examples of other components that may be encapsulated include SPF boosters, pigments, wetting agents, surfactants, stabilizers, other active ingredients, compounds that exhibit a cooling sensation (or other sensation) on the skin, etc.

In some cases, the capsules include one or more compounds that impart a cooling sensation (or other sensation such as warming, tingling, etc.) to the skin. For example, the one or more compounds that impart a cooling sensation to the skin may be substituted cyclohexanols and their esters, carboxamides, menthone ketals, menthoxypropanediols, etc. Non-limiting examples of compounds that impart a cooling sensation to the skin include menthol, isopulegol, menthyl lactate, N-ethyl-3-p-menthane carboxamide (WS-3), 2-isopropyl-N,2,3-trimethyl butanamide (WS-23), N-ethoxycarbonylmethyl-3-p-menthane carboxamide (WS-5), monomenthyl glutarate, monomenthyl succinate, and mixtures thereof.

The photosensitive capsules are typically used in sunscreen compositions, which often include, in addition to the capsules, a cosmetically acceptable carrier. The cosmetically acceptable carrier may be aqueous, non-aqueous, an alcohol, or an oil.

In some cases, the sunscreen composition is an emulsion, and therefore will typically include one or more emulsifiers (for example, gum Arabic).

In some embodiments, the sunscreen composition includes one or more film-formers (e.g., a copolymer of vinyl acetate and vinyl pyrrolidone), a booster, a pigment, a surfactant, a perfume, etc. Non-limiting example boosters include styrene/acrylates copolymer, calcium aluminum borosilicate, sodium borosilicate particulates, calcium/sodium borosilicate hollow microspheres, and calcium/sodium borosilicate microspheres.

The amount of photosensitive capsules used in a particular sunscreen composition can vary depending on the desired properties of the final product. For example, the sunscreen composition can include about 5, 10, 15, 20, 25, 30, 35, 40, 45, 50, 55, 60, 65, 70, 75, 80, or 85 wt. % of the capsules, based on the total weight of the sunscreen composition. The sunscreen composition can be formulated to include a range of capsules derived from any of the values set forth above (e.g., from about 15 wt. % to about 60 wt. %). Although the capsules typically include one or more UV filters, in some instances it may be desirable to have one or more UV filters in the sunscreen composition that are not encapsulated by the photosensitive capsules. For example, the sunscreen composition may include about 1, 2, 3, 4, or 5 wt. % to about 6, 7, 8, 9, 10, 15, 20, or 30 wt. %, based on the total weight of the sunscreen composition of one or more UV filters outside of the capsules. The sunscreen composition may include a combination of both capsules comprising one or more UV filters and additionally one or more UV filters that are not part of the capsules. In other instances, it may be desirable to exclude UV filters from the capsules and instead include other components in the capsules (e.g., boosters, compounds that impart a cooling sensation, etc.).

In some cases, the sunscreen composition may be transparent, pearlescent, or colored. Furthermore, the sunscreen composition may be in the form of a spray-on product, a lotion, a cream, a gel, a lipstick, a foundation, a moisturizer, a powder, etc.

The instant disclosure also relates to methods of using the photosensitive capsules and sunscreen compositions described herein. For example, the photosensitive capsules and sunscreen composition may be used in a method for protecting a keratinous substrate from UV radiation comprising applying a sunscreen composition of claim 7 to the keratinous substrate. In some cases, the keratinous substrate is skin or hair.

### Polymeric Photosensitive Degradable Shell

The polymeric photosensitive degradable shell is a polymer having photo-responsive groups that react in response to UV light to degrade the polymer. In other words, the polymeric photosensitive degradable shell “self-destructs” in response to UV light, thereby releasing the one or more UV filters and/or other components encapsulated by the photosensitive degradable shell.

In one embodiment, the polymeric photosensitive degradable shell comprises a polymer selected from the group consisting of a polyacrylate, a polyurea, a polyurethane, a polyester, a cellulose, a starch, a polysaccharide, and a silicone. These polymers can form a backbone to which photo-responsive groups are appended or the photo-responsive groups can be incorporated into the backbone of the polymer itself. The photo-responsive groups are chemical moieties that decompose or cause the polymer to deconstruct in response to UV light. For instance, non-limiting examples of photo-responsive groups include azobenzene groups, nitrobenzyl groups, a quinone-methide groups, o-nitrobenzyl groups, 6-nitro-veratroyloxycarbonyl (NVOC) groups, especially 6-nitro-veratroyloxycarbonyl groups having multiple methoxy groups (two methoxy groups or more) on the benzene ring, and di(nitrobenzyl)oxycarbonyl (DNBOC) groups. Additional examples of polymers having photo-responsive groups that react in response to UV light that can be used for the polymeric photosensitive degradable shell are described in Esser-Kahn et al., Triggered Release from Polymer Capsules, MACROMOLECULES, 44:5539-5553 (2011)), which is incorporated herein by reference in its entirety.

### UV Filters (Protective Agents)

The at least one UV filter can be any UV filter known in the art. A single UV filter may be used or a combination of UV filters may be used. For example, the UV filter may be one or more organic UV filters and/or one or more inorganic UV filters. Non-limiting examples of UV filters include:
i. Sparingly soluble UV filters (not appreciably soluble in either water or oil) such as Methylene Bis-Benzotriazolyl Tetramethylbutylphenol, Tris-Biphenyl Triazine, Methanone, 1,1′-(1,4-piperazinediyl)bis[1-[2-[4-(diethylamino)-2-hydroxybenzoyl]phen-yl]-and mixtures thereof.
ii. Oil soluble organic UV filters (at least partially soluble in oil or organic solvent), such as Bis-Ethylhexyloxyphenol Methoxyphenyl Triazine, Butyl Methoxydibenzoylmethane (BMBM), Oxybenzone, Sulisobenzone, Diethylhexyl Butamido Triazone (DBT), Drometrizole Trisiloxane, Ethylhexyl Methoxycinnamate (EHMC), Ethylhexyl Salicylate (EHS), Ethylhexyl Triazone (EHT), Homosalate, Isoamyl p-Methoxycinnamate, 4-Methylbenzylidene Camphor, Octocrylene (OCR), Polysilicone-15, and Diethylamino Hydroxy Benzoyl Hexyl Benzoate (DHHB);
iii. Inorganic UV filters such as titanium oxide and zinc oxide, iron oxide, zirconium oxide and cerium oxide; and
iv. Water soluble UV filters such as Phenylbenzimidazole Sulfonic Acid (PBSA), Sulisobenzone-sodium salt, Benzydilene Camphor Sulfonic Acid, Camphor Benzalkonium Methosulfate, Cinoxate, Disodium Phenyl Dibenzylmidazole Tetrasulfonate, Terephthalylidene Dicamphor Sulfonic Acid, PABA, and PEG-25 PABA.

In one embodiment, the UV filter is one or more UV filters selected from the group consisting of the UV filters provided in the table below:

| | TECHNICAL |
|---|---|
| INCI NAME | NAME |
| Octocrylene | Octocrylene |
| Butyl Methoxydibenzoylmethane | Avobenzone |
| Bis-EthylHexyloxyphenol Methoxyphenyl Triazine | Tinosorb S |
| Ethylhexyl Triazone | Uvinul T150 |
| Terephthalylidene Dicamphor Sulfonic Acid | Mexoryl SX |
| Drometrizole Trisiloxane | Mexoryl XL |
| Octyl salicylate or 2-ethylhexyl salicylate | Octisalate |
| 3,3,5-Trimethylcyclohexyl 2-hydroxybenzoate | Homosalate |

For example, in one embodiment the one or more UV filters is a combination being octocrylene, avobenzone, oxybenzone, octisalate, and homosalate. For instance, this combination of UV filters may be used in the following ratios relative to avobenzone:
the ratio of octocrylene to avobenzone is 1.6:1.0 to 2.4:1.0;
the ratio of oxybenzone to avobenzone 1.0:1.0 to 1.6:1.0;
the ratio of octisalate to avobenzone is 0.8:1.0 to 1.3:1.0; and
the ratio of homosalate to avobenzone is 2.8:1.0 to 4.3:1.

Furthermore, the ratio of each UV filter relative to avobenzone may be about: 2.0:1.0:1.3:1.1:3.6 (octocrylene:avobenzone:oxybenzone:octisalate:homosalate).

In another embodiment, the one or more UV filters is a combination of UV filters comprising octocrylene, avobenzone, octisalate, and homosalate, and optionally oxybenzone. For instance, this combination of UV filters may be used in the following ratios relative to avobenzone:
the ratio of octocrylene to avobenzone is 1.6:1.0 to 2.4:1.0,
the ratio of oxybenzone to avobenzone 0.0:1.0 to 0.016:1.0,
the ratio of octisalate to avobenzone is 1.3:1.0 to 2.0:1.0, and
the ratio of homosalate to avobenzone is 2.3:1.0 to 3.6:1.

Furthermore, the ratio of each UV filter relative to avobenzone may be about: 2.0:1.0:0.0:1.7:3.0 (octocrylene:avobenzone:oxybenzone:octisalate:homosalate).

In another embodiment, the one or more UV filters is a combination of UV filters comprising octocrylene, butyl methoxydibenzoylmethane, bis-ethylhexyloxyphenol methoxyphenyl triazine, ethylhexyl triazone, terephthalylidene dicamphor sulfonic acid, and drometrizole trisiloxane. For instance, this combination of UV filters may be used in the following ratios relative to butyl methoxydibenzoylmethane:
the ratio of octocrylene to butyl methoxydibenzoylmethane is 0.8:1.0 to 1.5:1.0;
the ratio of bis-ethylhexyloxyphenol methoxyphenyl triazine to butyl methoxydibenzoylmethane 0.3:1.0 to 0.8:1.0;
the ratio of ethylhexyl triazone to butyl methoxydibenzoylmethane is 0.3:1.0 to 1.0:1.0;
the ratio of terephthalylidene dicamphor sulfonic acid to butyl methoxydibenzoylmethane is 0.1:1.0 to 0.5:1.0; and
the ratio of drometrizole trisiloxane to butyl methoxydibenzoylmethane is 0.3:1.0 to 1.0:1.0.

Furthermore, the ratio of each UV filter relative to butyl methoxydibenzoylmethane may be about: 1.2:1.0:0.5:0.6:0.4:0.6 (octocrylene:butyl methoxydibenzoylmethane:bis-ethylhexyloxyphenol methoxyphenyl triazine:ethylhexyl triazone:terephthalylidene dicamphor sulfonic acid:drometrizole trisiloxane).

In another embodiment, the one or more UV filters is a combination of UV filters comprising octocrylene, butyl methoxydibenzoylmethane, ethylhexyl triazone, terephthalylidene dicamphor sulfonic acid, and drometrizole trisiloxane. For instance, this combination of UV filters may be used in the following ratios relative to butyl methoxydibenzoylmethane:
the ratio of octocrylene to butyl methoxydibenzoylmethane is 0.6:1.0 to 1.25:1.0;
the ratio of ethylhexyl triazone to butyl methoxydibenzoylmethane is 0.4:1.0 to 1.0:1.0;
the ratio of terephthalylidene dicamphor sulfonic acid to butyl methoxydibenzoylmethane is 0.3:1.0 to 0.7:1.0; and
the ratio of drometrizole trisiloxane to butyl methoxydibenzoylmethane is 0.4:1.0 to 1.1:1.0.

Furthermore, the ratio of each UV filter relative to butyl methoxydibenzoylmethane may be about: 1.0:1.0:0.7:0.5:0.7 (octocrylene:butyl methoxydibenzoylmethane:ethylhexyl triazone:terephthalylidene dicamphor sulfonic acid:drometrizole trisiloxane).

In another embodiment, the one or more UV filters is a combination of UV filters comprising octocrylene, butyl methoxydibenzoylmethane, bis-ethylhexyloxyphenol methoxyphenyl triazine, terephthalylidene dicamphor sulfonic acid, and drometrizole trisiloxane. For instance, this combination of UV filters may be used in the following ratios relative to butyl methoxydibenzoylmethane:
the ratio of octocrylene to butyl methoxydibenzoylmethane is 0.8:1.0 to 1.2:1.0;
the ratio of bis-ethylhexyloxyphenol methoxyphenyl triazine to butyl methoxydibenzoylmethane is 0.2:1.0 to 0.6:1.0;
the ratio of terephthalylidene dicamphor sulfonic acid to butyl methoxydibenzoylmethane is 0.0.25:1.0 to 0.75:1.0; and
the ratio of drometrizole trisiloxane to butyl methoxydibenzoylmethane is 0.4:1.0 to 0.8:1.0. [Synergistic combination from PR2012572]

Furthermore, the ratio of each UV filter relative to butyl methoxydibenzoylmethane may be about: 1.0:1.0:0.4:0.4:0.6 (octocrylene:butyl methoxydibenzoylmethane:bis-ethylhexyloxyphenol methoxyphenyl triazine:terephthalylidene dicamphor sulfonic acid:drometrizole trisiloxane).

In another embodiment, the one or more UV filters is a combination of UV filters comprising octocrylene, butyl methoxydibenzoylmethane, bis-ethylhexyloxyphenol methoxyphenyl triazine, terephthalylidene dicamphor sulfonic acid, and drometrizole trisiloxane. For instance, this combination of UV filters may be used in the following ratios relative to butyl methoxydibenzoylmethane is as follows:
the ratio of octocrylene to butyl methoxydibenzoylmethane is 0.8:1.0 to 1.3:1.0;
the ratio of bis-ethylhexyloxyphenol methoxyphenyl triazine to butyl methoxydibenzoylmethane is 0.1:1.0 to 0.6:1.0;
the ratio of ethylhexyl triazone to butyl methoxydibenzoylmethane is 0.2:1.0 to 0.6:1.0; and
the ratio of drometrizole trisiloxane to butyl methoxydibenzoylmethane is 0.3:1.0 to 0.7:1.0. [Synergistic combination from PR2012573]

Furthermore, the ratio of each UV filter relative to butyl methoxydibenzoylmethane may be about: 1.0:1.0:0.3:0.5:0.5 (octocrylene:butyl methoxydibenzoylmethane:bis-ethylhexyloxyphenol methoxyphenyl triazine:ethylhexyl triazone:drometrizole trisiloxane).

In another embodiment, the one or more UV filters is a combination of UV filters comprising octocrylene, butyl methoxydibenzoylmethane, bis-ethylhexyloxyphenol methoxyphenyl triazine, terephthalylidene dicamphor sulfonic acid, and terephthalylidene dicampohor sulfonic acid, which is incorporated herein by reference in its entirety. For instance, this combination of UV filters may be used in the following ratios relative to butyl methoxydibenzoylmethane is as follows:
the ratio of octocrylene to butyl methoxydibenzoylmethane is 0.8:1.0 to 1.6:1.0;
the ratio of bis-ethylhexyloxyphenol methoxyphenyl triazine to butyl methoxydibenzoylmethane is 0.2:1.0 to 0.6:1.0;
the ratio of ethylhexyl triazone to butyl methoxydibenzoylmethane is 0.3:1.0 to 0.6:1.0; and
the ratio of terephthalylidene dicampohor sulfonic acid to butyl methoxydibenzoylmethane is 0.01:1.0 to 0.3:1.0.

Furthermore, the ratio of each UV filter relative to butyl methoxydibenzoylmethane may be about: 1.2:1.0:0.3:0.5:0.1 (octocrylene:butyl methoxydibenzoylmethane:bis-ethylhexyloxyphenol methoxyphenyl triazine:ethylhexyl triazone:terephthalylidene dicampohor sulfonic acid).

In another embodiment, the one or more UV filters are pigments and/or nanopigments (mean size of the primary particles is generally is from 5 nm to 100 nm or from 10 nm to 50 nm) of treated or untreated metal oxides such as, for example, nanopigments of titanium oxide (amorphous or crystallized in rutile and/or anatase form), of iron oxide, of zinc oxide, of zirconium oxide or of cerium oxide. The treated nanopigments are pigments that have undergone one or more surface treatments of chemical, electronic, mechano-chemical and/or mechanical nature with compounds as described, for example, in Cosmetics & Toiletries, February 1990, Vol. 105, pp. 53-64, such as amino acids, beeswax, fatty acids, fatty alcohols, anionic surfactants, lecithins, sodium, potassium, zinc, iron or aluminum salts of fatty acids, metal (titanium or aluminum) alkoxides, polyethylene, silicones, proteins (collagen or elastin), alkanolamines, silicon oxides, metal oxides, sodium hexametaphosphate, alumina or glycerol. The treated nanopigments may more particularly be titanium oxides treated with:
silica and alumina, such as the products “Microtitanium Dioxide MT 500 SA” and “Microtitanium Dioxide MT 100 SA” from the company Tayca, and the products “Tioveil Fin”, “Tioveil OP”, “Tioveil MOTG” and “Tioveil IPM” from the company Tioxide;
alumina and aluminium stearate, such as the product “Microtitanium Dioxide MT 100 T” from the company Tayca;
alumina and aluminium laurate, such as the product “Microtitanium Dioxide MT 100 S” from the company Tayca;
iron oxides and iron stearate, such as the product “Microtitanium Dioxide MT 100 F” from the company Tayca;
silica, alumina and silicone, such as the products “Microtitanium Dioxide MT 100 SAS”, “Microtitanium Dioxide MT 600 SAS” and “Microtitanium Dioxide MT 500 SAS” from the company Tayca;
sodium hexametaphosphate, such as the product “Microtitanium Dioxide MT 150 W” from the company Tayca;
octyltrimethoxysilane, such as the product “T-805” from the company Degussa;
alumina and stearic acid, such as the product “UVT-M160” from the company Kemira;
alumina and glycerol, such as the product “UVT-M212” from the company Kemira;
alumina and silicone, such as the product “UVT-M262” from the company Kemira.

Other titanium oxide nanopigments treated with a silicone are TiO₂ treated with octyltrimethylsilane and for which the mean size of the elementary particles is between 25 and 40 nm, such as the product sold under the trade name “T805” by the company Degussa Silices, TiO₂ treated with a polydimethylsiloxane and for which the mean size of the elementary particles is 21 nm, such as the product sold under the trade name “70250 Cardre UF TiO2SI3” by the company Cardre, anatase/rutile TiO₂ treated with a polydimethylhydrogenosiloxane and for which the mean size of the elementary particles is 25 nm, such as the product sold under the trade name “Microtitanium Dioxide USP Grade Hydrophobic” by the company Color Techniques.

Uncoated titanium oxide nanopigments are sold, for example, by the company Tayca under the trade names “Microtitanium Dioxide MT 500 B” or “Microtitanium Dioxide MT 600 B”, by the company Degussa under the name “P 25”, by the company Wackher under the name “Oxyde de titane transparent PW”, by the company Myoshi Kasei under the name “UFTR”, by the company Tomen under the name “ITS” and by the company Tioxide under the name “Tioveil AQ”.

The uncoated zinc oxide nanopigments are, for example:
those sold under the name “Z-Cote” by the company Sunsmart;
those sold under the name “Nanox” by the company Elementis; and
those sold under the name “Nanogard WCD 2025” by the company Nanophase Technologies.

The coated zinc oxide nanopigments are, for example:
those sold under the name “Zinc Oxide CS-5” by the company Toshibi (ZnO coated with polymethylhydrogenosiloxane);
those sold under the name “Nanogard Zinc Oxide FN” by the company Nanophase Technologies (as a 40% dispersion in Finsolv TN, C₁₂-C₁₅ alkyl benzoate);
those sold under the name “Daitopersion ZN-30” and “Daitopersion ZN-50” by the company Daito (dispersions in cyclopolymethylsiloxane/oxyethylenated polydimethylsiloxane, containing 30% or 50% of nanozinc oxides coated with silica and polymethylhydrogenosiloxane);
those sold under the name “NFD Ultrafine ZNO” by the company Daikin (ZnO coated with perfluoroalkyl phosphate and copolymer based on perfluoroalkylethyl as a dispersion in cyclopentasiloxane);
those sold under the name “SPD-Z1” by the company Shin-Etsu (ZnO coated with silicone-grafted acrylic polymer, dispersed in cyclodimethylsiloxane);
those sold under the name “Escalol Z100” by the company ISP (alumina-treated ZnO dispersed in an ethylhexyl methoxycinnamate/PVP-hexadecene/methicone copolymer mixture);
those sold under the name “Fuji ZNO-SMS-10” by the company Fuji Pigment (ZnO coated with silica and polymethylsilsesquioxane); and
those sold under the name “Nanox Gel TN” by the company Elementis (ZnO dispersed at a concentration of 55% in C₁₂-C₁₅ alkyl benzoate with hydroxystearic acid polycondensate).

The uncoated cerium oxide nanopigments are sold under the name “Colloidal Cerium Oxide” by the company Rhone-Poulenc. The uncoated iron oxide nanopigments are sold, for example, by the company Arnaud under the names “Nanogard WCD 2002 (FE 45B)”, “Nanogard Iron FE 45 BL AQ”, “Nanogard FE 45R AQ” and “Nanogard WCD 2006 (FE 45R)” or by the company Mitsubishi under the name “TY-220”. The coated iron oxide nanopigments are sold, for example, by the company Arnaud under the names “Nanogard WCD 2008 (FE 45B FN)”, “Nanogard WCD 2009 (FE 45B 556)”, “Nanogard FE 45 BL 345” and “Nanogard FE 45 BL” or by the company BASF under the name “Transparent Iron Oxide”.

Mixtures of metal oxides may also be used, especially of titanium dioxide and of cerium dioxide, including the silica-coated equal-weight mixture of titanium dioxide and of cerium dioxide, sold by the company Ikeda under the name “Sunveil A”, and also the alumina, silica and silicone-coated mixture of titanium dioxide and of zinc dioxide, such as the product “M 261” sold by the company Kemira, or the alumina, silica and glycerol-coated mixture of titanium dioxide and of zinc dioxide, such as the product “M 211” sold by the company Kemira. Additional UV filters and combinations of UV filters that can further be used are described in U.S. Pat. No. 8,557,227, U.S. Pat. No. 8,652,449, and U.S. Pat. No. 8,691,192; and US Patent Application Publication Nos. US 2010/0129303, US 2013/0129649, US 20130129650, US 20140170093, US 20140170094, which are incorporated herein by reference in their entirety.

The one or more UV filter may be in an amount of about 1, 5, 6, 7, 8, 9, or 10 to about 15, 20, 25, 30, 35, 40, 45, 50, 55, 60, 65, 70, 75, 80, 85, 90 or 90 wt. %, based on the total weight of the capsule, or alternatively, based on the total weight of the sunscreen composition. In other embodiments, the at least one UV filter may be present in a positive amount but not in excess of (no more than) about 5, 6, 7, 8, 9, 10, 11, 12, 13, 14, 15, 16, 17, 18, 19, 20, 21, 22, 23, 24, 25, 26, 27, 28, 29, or 30 wt. %, based on the total weight of the composition. The sunscreen compositions according to the instant disclosure may be formulated to have an SPF of 5, 10, 15, 20, 25, 30, 35, 40, 45, 50, 55, 60, 65, 70, 75, 80, 85, 90, 95, 100, 105, 110, 115, 125, 130, 150, 175, or 200. Likewise, the SPF of the sunscreen composition may be formulated to fall within a range between any of the SPF values provided in the previous sentences (inclusive of the values).

### Boosters

The term “booster” or “SPF booster” means a compound or composition that when used in a formulation in conjunction with a UV filtering agent, increases the SPF of the formulation without increasing the amount of UV filtering agent in the formulation. The at least one booster can be any booster known in the art. For example, the at least one booster may be selected from the group consisting of styrene/acrylates copolymer (Sunspheres®), calcium aluminum borosilicate, sodium borosilicate particulates, calcium/sodium borosilicate hollow microspheres, and calcium/sodium borosilicate microspheres. In one embodiment, the booster is styrene/acrylates copolymer (Sunspheres®).

Other examples of boosters are those capable of reflecting UV light such as glass microspheres. Typically, the glass microspheres used in the compositions are essentially homogeneous and essentially uniform in sphericity and have a mean particle size of between about 5 μm and 70 μm, such as from about 10 μm to 20 μm. Glass microspheres useful in the present invention include hollow microspheres of calcium aluminum borosilicate (commercially available from Presperse Inc. under the trade name LUXSIL®), sodium borosilicate particulates (commercially available from PQ Corporation under the trade name Q-CEL 570), calcium/sodium borosilicate hollow microspheres (commercially available from 3M under the trade names ES 22 and 1 K), calcium/sodium borosilicate microspheres (commercially available from 3M's under the trade name Scotchlite™ K₂₀ product).

The compositions may include one or more boosters. The booster itself is not typically an active ingredient (i.e., UV filter), but is designed to enhance the effectiveness of the sunscreen actives present in the formulation. Suitable boosters include, but are not limited to, styrene/acrylates copolymer, sodium bentonites, highly purified white sodium bentonites, montmorillonite, fluorene derivatives, ester derivatives of cyano(9H-fluoren-9-ylidene), amides, malates, bis-urethanes, or any combinations thereof.

### Emulsifiers

The sunscreen compositions typically include at least one emulsifier such as an amphoteric, anionic, cationic or nonionic emulsifier, used alone or as a mixture, and optionally a co-emulsifier. The emulsifiers are chosen in an appropriate manner according to the emulsion to be obtained (W/O or O/W). The emulsifier and the co-emulsifier are generally present in the composition in a proportion ranging from 0.3% to 30% by weight and preferably from 0.5% to 20% by weight relative to the total weight of the composition.

For W/O emulsions, examples of emulsifiers that may be mentioned include dimethicone copolyols, such as the mixture of cyclomethicone and dimethicone copolyol sold under the trade name DC 5225 C by the company Dow Corning, and alkyl dimethicone copolyols such as the lauryl dimethicone copolyol sold under the name Dow Corning 5200 Formulation Aid by the company Dow Corning, and the cetyl dimethicone copolyol sold under the name Abil EM90™ by the company Goldschmidt. A cross-linked elastomeric solid organopolysiloxane comprising at least one oxyalkylene group, such as those obtained according to the procedure of Examples 3, 4 and 8 of U.S. Pat. No. 5,412,004 and of the examples of U.S. Pat. No. 5,811,487, especially the product of Example 3 (synthesis example) of U.S. Pat. No. 5,412,004, such as the product sold under the reference KSG 21 by the company Shin-Etsu, may also be used as surfactants for W/O emulsions.

For O/W emulsions, examples of emulsifiers that may be mentioned include nonionic emulsifiers such as oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters; oxyalkylenated (oxyethylenated and/or oxypropylenated) fatty alcohol ethers; sugar esters such as sucrose stearate; and mixtures thereof.

The fatty acid esters of a sugar that can be used as nonionic amphiphilic lipids can be chosen in particular from the group comprising esters or mixtures of esters of a C₈-C₂₂ fatty acid and of sucrose, of maltose, of glucose or of fructose, and esters or mixtures of esters of a C₁₄-C₂₂ fatty acid and of methylglucose.

The C₈-C₂₂ or C₁₄-C₂₂ fatty acids forming the fatty unit of the esters that can be used in the emulsion comprise a saturated or unsaturated linear alkyl chain having, respectively, from 8 to 22 or from 14 to 22 carbon atoms. The fatty unit of the esters can be chosen in particular from stearates, behenates, arachidonates, palmitates, myristates, laurates, caprates and mixtures thereof.

By way of example of esters or of mixtures of esters of a fatty acid and of sucrose, of maltose, of glucose or of fructose, mention may be made of sucrose monostearte, sucrose distearate, sucrose tristearate and mixtures thereof, such as the products sold by the company Croda under the name Crodesta F50, F70, F110 and F160 having, respectively, an HLB (Hydrophilic Lipophilic Balance) of 5, 7, 11 and 16; and, by way of example of esters or of mixtures of esters of a fatty acid and of methylglucose, mention may be made of the disearate of methylglucose and of polyglycerol-3, sold by the company Goldschmidt under the name Tego-care 450. Mention may also be made of glucose monoesters or maltose monoesters, such as methyl O-hexadecanoyl-6-D-glucoside and O-hexadecanoyl-6-D-maltoside.

The fatty alcohol ethers of a sugar that can be used as nonionic amphiphilic lipids can be chosen in particular form the group comprising ethers or mixtures of ethers of a C₈-C₂₂ fatty alcohol and of glucose, of maltose, of sucrose or of fructose, and ethers or mixtures of ethers of a C₁₄-C₂₂ fatty alcohol and of methylglucose. They are in particular alkylpolyglucosides.

The C₈-C₂₂ or C₁₄-C₂₂ fatty alcohols forming the fatty unit of the ethers that can be used in the emulsion of the instant disclosure comprise a saturated or unsaturated linear alkyl chain having, respectively, from 8 to 22 or from 14 to 22 carbon atoms. The fatty unit of the ethers can be chosen in particular from decyl, cetyl, behenyl, arachidyl, stearyl, palmityl, myristyl, lauryl, capryl and hexadecanoyl units, and mixtures thereof such as cetearyl.

By way of example of fatty alcohol ethers of a sugar, mention may be made of alkylpolyglucosides, such as decylglucoside and laurylglucoside sold, for example, by the company Henkel under the respective names Plantaren 2000 and Plantaren 1200, cetostearylglucoside, optionally as a mixture with cetostearyl alcohol, sold, for example, under the name Montanov 68 by the company Seppic, under the name Tego-care CG90 by the company Goldschmidt and under the name Emulgade KE3302 by the company Henkel, and also arachidylglucoside, for example in the form of the mixture of arachidyl and behenyl alcohols and of arachidylglucoside sold under the name Montanov 202 by the company Seppic.

Use is more particularly made, as nonionic amphiphilic lipid of this type, of sucrose monostearate, sucrose distearate, sucrose tristearate and mixtures thereof, the distearate of methylglucose and of polyglycerol-3, and alkylpolyglucosides.

The glycerol fatty esters that can be used as nonionic amphiphilic lipids can be chosen in particular from the group comprising the esters formed from at least one acid comprising a saturated linear alkyl chain having from 16 to 22 carbon atoms, and from 1 to 10 glycerol units. Use may be made of one or more of these glycerol fatty esters in the emulsion of the instant disclosure.

These esters may be chosen in particular from stearates, behenates, arachidates, palmitates and mixtures thereof. Stearates and palmitates are preferably used.

By way of example of a surfactant that can be used in the emulsion of the instant disclosure, mention may be made of decaglycerol monostearate, distearate, tristearate and pentastearate (10 glycerol units) (CTFA names: polyglyceryl-10 stearate, polyglyceryl-10 distearate, polyglyceryl-10 tristearate, polyglyceryl-10 pentastearate), such as the products sold under the respective names Nikkol Decaglyn 1-S, 2-S, 3-S and 5-S by the company Nikko, and diglyceryl monostearate (CTFA name: polyglyceryl-2 stearate) such as the product sold by the company Nikko under the name Nikkol DGMS.

The sorbitan fatty esters that can be used as nonionic amphiphilic lipids chosen in particular from the group comprising esters of a C₁₆-C₂₂ fatty acid and of sorbitan and oxyethylenated esters of a C₁₆-C₂₂ fatty acid and of sorbitan. They are formed from at least one fatty acid comprising at least one saturated linear alkyl chain, having, respectively, from 16 to 22 carbon atoms, and from sorbitol or from ethoxylated sorbitol. The oxyethylenated esters generally comprise from 1 to 100 ethylene oxide units, and preferably from 2 to 40 ethylene oxide (EO) units.

These esters can be chosen in particular from stearates, behenates, arachidates, palmitates and mixtures thereof. Stearates and palmitates are preferably used.

By way of example of sorbitan fatty ester and of an oxyethylenated sorbitan fatty ester, mention may be made of sorbitan monostearate (CTFA name: sorbitan stearate) sold by the company ICI under the name Span 60, sorbitan monopalmitate (CTFA name: sorbitan palmitate) sold by the company ICI under the name Span 40, or sorbitan 20 EO tristearate (CTFA name: polysorbate 65) sold by the company ICI under the name Tween 65.

The ethoxylated fatty ethers are typically ethers made up of 1 to 100 ethylene oxide units and of at least one fatty alcohol chain having from 16 to 22 carbon atoms. The fatty chain of the ethers can be chosen in particular from behenyl, arachidyl, stearyl and cetyl units, and mixtures thereof, such as cetearyl. By way of example of ethoxylated fatty ethers, mention may be made of ethers of behenyl alcohol comprising 5, 10, 20 and 30 ethylene oxide units (CTFA names: beheneth-5, beheneth-10, beheneth-20 and beheneth-30), such as the products sold under the names Nikkol BBS, BB10, BB20 and BB30 by the company Nikko, and the ether of stearyl alcohol comprising 2 ethylene oxide units (CTFA name: steareth-2), such as the product sold under the name Brij 72 by the company ICI.

The ethoxylated fatty esters that can be used as nonionic amphiphilic lipids are esters made up of 1 to 100 ethylene oxide units and of at least one fatty acid chain comprising from 16 to 22 carbon atoms. The fatty chain of the esters can be chosen in particular from stearate, behenate, arachidate and palmitate units, and mixtures thereof. By way of example of ethoxylated fatty esters, mention may be made of the ester of stearic acid comprising 40 ethylene oxide units, such as the product sold under the name Myrj 52 (CTFA name: PEG-40 stearate) by the company ICI, and the ester of behenic acid comprising 8 ethylene oxide units (CTFA name: PEG-8 behenate), such as the product sold under the name Compritol HD5 ATO by the company Gattefosse.

The block copolymers of ethylene oxide and of propylene oxide that can be used as nonionic amphiphilic can be chosen in particular from poloxamers and in particular from Poloxamer 231, such as the product sold by the company ICI under the name Pluronic L81 of formula (V) with x=z=6, y=39 (HLB 2); Poloxamer 282, such as the product sold by the company ICI under the name Pluronic L92 of formula (V) with x=z=10, y=47 (HLB 6); and Poloxamer 124, such as the product sold by the company ICI under the name Pluronic L44 of formula (V) with x=z=11, y=21 (HLB 16).

As nonionic amphiphilic lipids, mention may also be made of the mixtures of nonionic surfactants described in document EP-A-705593, incorporated herein for reference.

Suitable hydrophobically-modified emulsifiers include, for example, inulin lauryl carbamate, commercially available from Beneo Orafti under the tradename Inutec SP1.

The above lists are only examples and not limiting.

The total amount of emulsifier present in the compositions is typically in an amount of about 0.1, 0.2, or 0.5 wt. % to about 4.0, 5.0, 6.0, or 7.5 wt. %, based on the total weight of the composition.

### Gelling Agents

Examples of suitable hydrophilic gelling agents that may be used in the instant compositions include carboxyvinyl polymers such as the Carbopol products (carbomers) and the Pemulen products (acrylate/C10-C30-alkylacrylate copolymer); polyacrylamides, for instance the cross-linked copolymers sold under the names Sepigel 305 (CTFA name: polyacrylamide/C13-14 isoparaffin/Laureth 7) or Simulgel 600 (CTFA name: acrylamide/sodium acryloyldimethyltaurate copolymer/isohexadecane/polysorbate 80) by the company SEPPIC; 2-acrylamido-2-methylpropanesulfonic acid polymers and copolymers, which are optionally cross-linked and/or neutralized, for instance the poly(2-acrylamido-2-methylpropanesulfonic acid) sold by the company Hoechst under the trade name “Hostacerin AMPS” (CTFA name: ammonium polyacryldimethyltauramide); cellulose-based derivatives such as hydroxyethylcellulose; polysaccharides and especially gums such as xanthan gum; and mixtures thereof.

Lipophilic gelling agents (thickeners) that may be mentioned include modified clays such as hectorite and its derivatives, for instance the products sold under the name bentone.

In some instances, the gelling agent is ammonium acryloyldimethyltaurate/steareth-25 methacrylate crosspolymer, commercially available from Clariant under the tradename Aristoflex HMS.

Bis-Methoxy PEG-13 PEG-438/PPG-110 SMDI copolymer is a copolymer of PEG-438/PPG-110 and saturated methylene diphenyldiisocyanate (SMDI) monomers, end-capped with methoxy PEG-13.

The above lists are only examples and not limiting.

The gelling agent is typically used in an amount of about 0.05 to about 1.5% by weight, from about 0.08 to about 1.0% by weight, or about 0.1 to about 0.5% by weight, based on the total weight of the composition.

### Wetting Agents

Examples of wetting agents that may be used in the instant compositions included dimethicone copolyol compounds such as PEG-12 dimethicone available from Dow Corning®.

### Oils/Emollients

Examples of oils/emollients that may be included in the sunscreen compositions include: hydrocarbon-based oils of plant origin, such as liquid triglycerides of fatty acids containing from 4 to 10 carbon atoms, for instance heptanoic or octanoic acid triglycerides, or alternatively, for example, sunflower oil, corn oil, soybean oil, marrow oil, grapeseed oil, sesameseed oil, hazelnut oil, apricot oil, macadamia oil, arara oil, coriander oil, castor oil, avocado oil, caprylic/capric acid triglycerides, for instance those sold by the company Stearineries Dubois or those sold under the names Miglyol 810, 812 and 818 by the company Dynamit Nobel, jojoba oil, shea butter oil and caprylyl glycol; synthetic esters and ethers, especially of fatty acids, for instance Purcellin oil, 2-octyldodecyl stearate, 2-octyldodecyl erucate, isostearyl isostearate and ethylhexyl palmitate; hydroxylated esters, for instance isostearyl lactate, octyl hydroxystearate, octyldodecyl hydroxystearate, diisostearyl malate or triisocetyl citrate; fatty alcohol heptanoates, octanoates or decanoates; polyol esters, for instance propylene glycol dioctanoate, neopentyl glycol diheptanoate and diethylene glycol diisononanoate; and pentaerythritol esters, for instance pentaerythrityl tetraisostearate, or isopropyl lauroyl sarcosinate, sold especially under the trade name Eldew SL 205 by the company Ajinomoto; linear or branched hydrocarbons, of mineral or synthetic origin, such as volatile or non-volatile liquid paraffins, and derivatives thereof, petroleum jelly, polydecenes, isohexadecane, isododecane, hydrogenated polyisobutene such as Parleam oil, or the mixture of n-undecane (C₁₁) and of n-tridecane (C₁₃) sold under the reference Cetiol UT by the company Cognis; fluoro oils that are partially hydrocarbon-based and/or silicone-based, for instance those described in document JP-A-2 295 912; silicone oils, for instance volatile or non-volatile polymethylsiloxanes (PDMS) with a linear or cyclic silicone chain, which are liquid or pasty at room temperature, in particular volatile silicone oils, especially cyclopolydimethylsiloxanes (cyclomethicones) such as cyclohexadimethylsiloxane and cyclopentadimethylsiloxane; polydimethylsiloxanes comprising alkyl, alkoxy or phenyl groups, which are pendent or at the end of a silicone chain, these groups containing from 2 to 24 carbon atoms; phenyl silicones, for instance phenyl trimethicones, phenyl dimethicones, phenyltrimethylsiloxydiphenylsiloxanes, diphenyl dimethicones, diphenylmethyldiphenyltrisiloxanes or 2-phenylethyl trimethylsiloxy silicates, and polymethylphenylsiloxanes; mixtures thereof.

Additional examples include benzoic acid esters of C₉-C₁₅ alcohols, isononyl iso-nonanoate, C₁₂-C₁₅ alkyl benzoate, or any combinations thereof.

Specific examples of oils/emollients include ethylhexyl palmitate, cocoglyceride, cyclomethicone, dimethicone, dicapryl maleate, caprylic/capric triglyceride, isopropyl myristate, octyl stearate, isostearyl linoleate, lanolin oil, coconut oil, cocoa butter, olive oil, avocado oil, aloe extracts, jojoba oil, castor oil, fatty acid, oleic acid, stearic acid, fatty alcohol, cetyl alcohol, hexadecyl alcohol, diisopropyl adipate, hydroxybenzoate esters, benzoic acid esters of C₉-C₁₅ alcohols, isononyl iso-nonanoate, alkanes, mineral oil, silicone, dimethyl polysiloxane, ether, polyoxypropylene butyl ether, polyoxypropylene cetyl ether, C₁₂-C₁₅ alkyl benzoate, aryl alkyl benzoate, Isopropyl Lauroyl sarcosinate, and any combinations thereof. In some cases, a preferred emollient is ethylhexy palmitate.

Examples of hydrophilic organic solvents that may be included in the sunscreen compositions include:
monohydric C₁-C₈ alcohols such as ethanol, propanol, butanol, isopropanol, isobutanol;
Polyethylene glycols from 6 to 80 ethylene oxides such as propylene glycol, isoprene glycol, butylene glycol, glycerol, sorbitol;
mono or di-alkyl isosorbides such as dimethyl isosorbide;

Examples of amphiphilic organic solvents include: polypropylene glycol (PPG) like propylene glycol alkyl ester or alkyl ether of PPG like PPG-23 oleyl ether and PPG-36 oleate.

The above lists are only examples and not limiting.

The total amount of oils/emollient present in the compositions is typically about 0.1, 0.5, 1.0, or 2.5 wt. % to about 5.0, 7.5, 10.0, 15.0, 20.0, 30.0, or 40 wt. % of the total weight of the composition.

Film Formers

Film-formers are often incorporated into sunscreen compositions to ensure even coverage of the UV filters and can be used to render the composition water resistant. The film former is typically a hydrophobic material that imparts film forming and/or waterproofing characteristics. One such agent is polyethylene, which is available from New Phase Technologies as Performalene® 400, a polyethylene having a molecular weight of 400. Another suitable film former is polyethylene 2000 (molecular weight of 2000), which is available from New Phase Technologies as Performalene®. Yet, another suitable film former is synthetic wax, also available from New Phase Technologies as Performa® V-825. Other typical film-formers include acrylates/acrylamide copolymer, acrylates copolymer, acrylates/C₁₂-C₂₂ alkylmethacrylate copolymer, polyethylene, waxes, VP/dimethiconylacrylate/polycarbamylpolyglycol ester, butylated PVP, PVP/hexadecene copolymer, octadecene/MA copolymer, PVP/eicosene copolymer, tricontanyl PVP, Brassica Campestris/Aleuritis Fordi Oil copolymer, decamethyl cyclopentasiloxane (and) trimethylsiloxysilicate, and mixtures thereof. In some cases, the film former is acrylates/C₁₂-C₂₂ alkylmethacrylate copolymer sold under the tradename Allianz OPT® by ISP.

Many of the common film-forming polymers included in sunscreen compositions are not soluble in ethanol (such as PVP/Eicosene copolymer). A common film-former employed in ethanol based sunscreen products is Dermacryl LT or Dermacryl 79 marketed by Akzo Nobel (INCI Name: acrylates/octylacrylamide copolymner). Dermacryl LT (CAS Number: 80570-62-3) is a hydrophobic, high molecular weight carboxylated acrylic copolymer. It functions as a film-former in a broad range of cosmetic formulations, imparting waterproofing, increased occlusivity and decreased rub-off of actives.

The above lists are only examples and not limiting.

The total amount of film-formers present in the compositions is typically in an amount of about 0.1, 0.5, 1.0, or 5 wt. % to about 5, 10, 20, or 25 wt. %, based on the total weight of the composition.

### Methods of Manufacture

The photosensitive capsules may be formed by any method known in the art. For instance, the capsules may be prepared by colloidosome formation, polymer precipitation by phase separation, interfacial polymerization, layer-by-layer polyelectrolyte deposition, polymer growth by surface polymerization, and copolymier vesicle formation. Each of these methods has different characteristics. Therefore, the choice of an appropriate method depends on the properties of the shell material, the encapsulated material, and the final sunscreen formulation for the capsules. Photosensitive capsules can be manufactures, for example, as described in Dispinar et al, Polyurea Microcapsules with a photocleavable Shell: UV-Triggered Release, POLYM. CHEM 4:763 (2013) and Fomina et al., UV and Near-IR Triggered Release from Poymeric Nanopraticles, J. AM. CHEM. SOC., 132:9540-9542 (2010), both of which are incorporated herein by reference in their entirety.

The compositions according to the instant disclosure may be in the form of an emulsion, e.g., oil-in-water or water-in-oil type. They may be in particular in the form of a simple or complex emulsion (O/W, W/O, O/W/O or W/O/W emulsion) such as a cream or a milk, in the form of a gel or a cream-gel, or in the form of a lotion. The sunscreen composition may be clear or transparent. The sunscreen composition may be formulated as a spray-on product; and in some instances, the sunscreen composition is a clear, spray-on product.

The instant disclosure will be better understood from the examples that follow, all of which are intended for illustrative purposes only and are not meant to limit the scope of the instant disclosure in any way.

### EXAMPLES

### Example 1

### Noncapsules Containing Mexoryl XL

Nanocapsules having a polymeric photosensitive degradable shell and multiple photo-response groups can be synthesized by incorporation of quinone-methide moieties into a polymeric shell, as shown below.

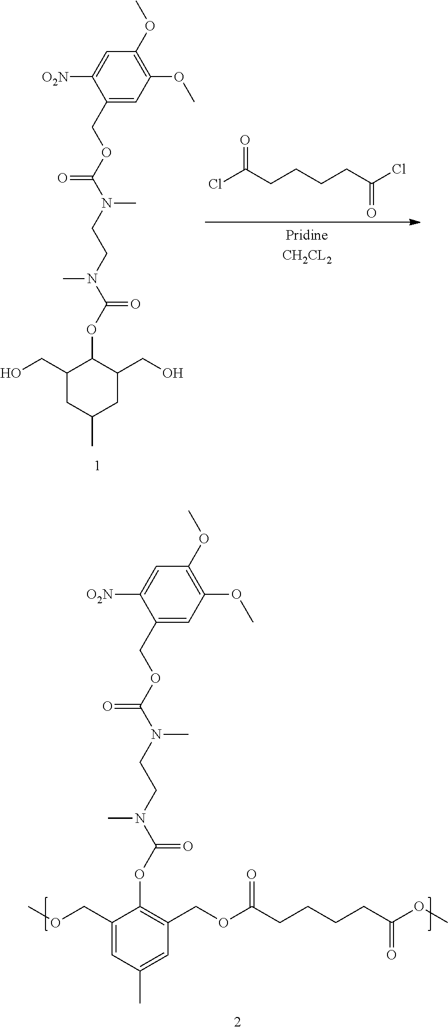

Monomer 1 can be synthesized according to the procedures set forth in Amir et al., Angew. Chem., Int, Ed., 42:4494-4499 (2003), which is incorporated herein by reference in its entirety. 4,5-dimethoxy-2-nitrobenzyl alcohol is shown here but 6-bromo-coumarins or fluorine-based compounds can also be used. Monomer 1 is copolymerized with adipoyl choloride to yield a regular copolymer. The low molecular weight oligomers are removed by repeated precipitation of the crude polymer with cold ethanol, to yield a final product with a molecular weight of about 65,000 Da.

Nanocapsules are formed using a single emulsion method and encapsulating drometrizole trisiloxane (Mexoryl XL), or one or more other UV filters. The average diameter size of the nanocapsules is about 170 nm. The release of the one or more UV filters from the nanocapsules can be observed by fluorescence spectroscopy. Upon irradiation with 350 nm light, the fluorescence intensity drops, indicating burst release of the one or more UV filters.

### Example 2

### Microcapsules Containing Mexoryl XL

Microcapsules having a polymeric photosensitive degradable shell and multiple photo-response groups can be synthesized using 6-nitro-veratroyloxycarbonyl (NVOC)-based diisocyanate oligmer. For the preparation of the NVOC-based diisocyanate oligomer, a photolabile diol is first synthesized from commercially available acetovanillone in a three-step procedure. First, the acetovanillone is alkylated with methyl-4-bromoacetate and subsequently nitrated with fuming nitric acid according. Then, the ketone and methyl ester groups are reduced with sodium borohydride in a MeOH—THF system to yield the photolabile diol. In order to obtain the NVOC-based diisocyanate oligomer, the photolabile diol is polymerized with toluene 2,4-diisocyanate in anhydrous ethyl acetate at 70° C. in the presence of dibutyltin dilaurate catalyst. An excess amount of toluene 2,4-diisocyanate is used in the polymerization because it ensures the isocyanate end functionality and because a low molecular weight oligomer can be obtained. Synthesis of low molecular weight diisocyanate oligomers is targeted to avoid potential solubility problems as a result of the rigid-urethane structure during the microcapsule synthesis. Furthermore, a low molecular weight diisocyanate oligomer provides a high isocyanate content during the microcapsule synthesis, consequently leading to the formation of a robust shell.

Polyurea microcapsules loaded with drometrizole trisiloxane (Mexoryl XL) can be prepared by interfacial polymerization in a stable oil-in-water emulsion system. First, the NVOC-based diisocyanate oligomer and the Mexoryl XL are dissolved in chlorobenzene to prepare the oil phase. The oil phase is then poured into an aqueous solution of a gum arabic surfactant to make an oil-in-water emulsion using a vortex mixer. An aqueous solution of diethylenetriamine (DETA) is added drop-wise to the emulsion in order to begin the encapsulation process. The reaction between the amino groups of the DETA in the aqueous phase and the isocyanate end groups of the NVOC-based oligomer in the oil phase produces a polyuria membrane around the oil droplets that restricts the diffusion of the unreacted DETA. Thus, for increased stabilization of the shell, the emulsion can be cured in an oil bath at 80° C. for one hour. Microcapsules are obtained by successive washing and drying steps.

The microcapsules are in the range of 50 to 300 μm in diameter (mean value of about 180 μm), which can be confirmed by particle size analysis. When exposed to UV light, the photo-responsive groups are activated and polymeric photosensitive degradable shell breaks apart and releases the UV filter.

### Example 3

### Sunscreen Composition (Oil-in-Water Emulsion)

| Phase | US INCl name | O/W emulsion |
|---|---|---|
| A | Water | Q.S. |
| | EDTA | 0.1 |
| | Propylene Glycol | 2.5 |
| | Preservatives | 0.8 |
| B | Ethylhexyl Palmitate | 5 |
| | Isohexadecane | 7.5 |
| | Glyceryl Stearate (and) PEG-100 | 2 |
| | Stearate | |
| | Stearyl Alcohol | 0.5 |
| | Dimethicone | 1.7 |
| C | Carbomer | 0.2 |
| D | Nanocapsules of Example 1 | 40 |
| | Microcapsules of Example 2 | |

The components of Phase A are combined and heated to 70-75° C. The components of Phase B are separately combined and heated to 70-75° C. Phase C (Carbomer) is added to Phase B and mixed for 15 minutes. This mixture is then added into Phase A and homogenized for 20 minutes. The product is cooled to room temperature and then Phase D is added with slow mixing for about 10 minutes.

### Example 4

### Sunscreen Composition (Water-in-Oil Emulsion)

| Phase | US INCl name | W/O emulsion |
|---|---|---|
| A | Water | Q.S. |
| | EDTA | 0.1 |
| | Propylene Glycol | 2.5 |
| | Preservatives | 0.8 |
| | Sodium Chloride | 0.5 |
| B | Ethylhexyl Palmitate | 5 |
| | Isohexadecane | 7.5 |
| | Lauryl PEG/PPG-18-18 Methicone | 2 |
| | Peg-30 Dipolyhydroxystearate | 3 |
| | Dimethicone | 1.7 |
| C | Nanocapsules of Examples 1 or | 40 |
| | Microcapsules of Example 2 | |

The components of Phase A are combined and heated to 70-75° C. The components of Phase B are separately combined and heated to 70-75° C. Phase A is then added to Phase B and the combination is homogenized for 20 minutes. After the homogenized product is cooled to room temperature, Phase C is add with slow mixing.

### Example 5

### Sunscreen Composition (Alcohol Based)

| Phase | US INCl name | Alcoholic |
|---|---|---|
| A | Ethanol | Q.S. |
| B | Acrylate/Octylacrylamide Copolymer | 2 |
| C | Ethylhexyl Palmitate | 10 |
| D | Nanocapsules of Example 1 or | 40 |
| | Microcapsules of Example 2 | |

Phase A is placed in a beaker at room temperature. While mixing Phase B is slowly added to Phase A and mixed until clear. Phase C is added to the mixture of Phases A and B and mixed at room temperature. Finally, Phase D is added with low mixing.

### Example 6

### Sunscreen Composition (Oil Based)

| Phase | US INCl name | Oil FLA |
|---|---|---|
| A | Ethylhexyl Palmitate | 15 |
| | Octyl-2 decanol | 10 |
| | Dicaprylyl Carbonate | 15 |
| | Dicaprylyl Ether | 15 |
| B | Nanocapsules of Example 1 | 30 |
| | Microcapsules of Example 2 | |
| | total: | 100 |

The components of Phase A are combined and mixed at room temperatures. The components of Phase B then are then mixed into Phase A.

1. Photosensitive capsules comprising:
(i) a polymeric photosensitive degradable shell; and
(ii) one or more UV filters encapsulated by the photosensitive degradable shell,
wherein the one or more UV filters is released from the photosensitive degradable shell upon exposure to UV light.

2. The photosensitive capsules of claim 1, wherein the (i) polymeric photosensitive degradable shell comprises a polymer having photo-responsive groups that react in response to UV light to degrade the polymer, thereby releasing the one or more UV filters from the capsules.

3. The photosensitive capsules of claim 1, wherein the (i) polymer having photo-responsive groups that react in response to UV light is selected from the group consisting of a polyacrylate, a polyurea, a polyurethane, polyester, cellulose, starch, a polysaccharide, and a silicone.

4. The photosensitive capsules of claim 2, wherein the photo-responsive groups are selected from the group consisting of an azobenzene group, a nitrobenzyl group, a quinone-methide group, and an o-nitrobenzyl group.

5. The photosensitive capsules of claim 4, wherein the photo-responsive group is a 6-nitro-veratroyloxycarbonyl groups containing two methoxy groups on the benzene ring.

6. The photosensitive capsules of claim 1, wherein the (ii) one or more UV filters includes drometrizole trisiloxane.

7. The photosensitive capsules of claim 1 having an average diameter size of about 50 nm to about 850 μm.

8. The capsules of claim 1, wherein 50% or more of the one or more UV filters is released from the photosensitive degradable shell within 30 minutes upon exposure to UV light having a wavelength of 350 nm.

9. The capsules of claim 1, further comprising:
(iii) one or more compounds that impart a cooling sensation to the skin.

10. (canceled)

11. (canceled)

12. Photosensitive capsule comprising:
(i) about 10 wt. % to about 60 wt. % of a polymeric photosensitive degradable shell; and
(ii) about 10 wt. % to about 75 wt. % of one or more UV filters encapsulated by the photosensitive degradable shell,
wherein the percent by weight is based on the total weight of the particle.

13. A sunscreen composition comprising:
(a) capsules of claim 1; and
(b) a cosmetically acceptable carrier.

14. The sunscreen composition of claim 13, wherein the (b) cosmetically acceptable carrier is aqueous.

15. The sunscreen composition of claim 13, wherein the (b) cosmetically acceptable carrier is non-aqueous.

16. (canceled)

17. The sunscreen composition of claim 13, wherein the composition is an emulsion and further comprises:
(c) one or more emulsifiers.

18. (canceled)

19. The sunscreen composition of claim 18, further comprising:
(d) one or more film-formers.

20. The sunscreen composition of claim 13, further comprising:
(e) a booster selected from the group consisting of styrene/acrylates copolymer, calcium aluminum borosilicate, sodium borosilicate particulates, calcium/sodium borosilicate hollow microspheres, and calcium/sodium borosilicate microspheres.

21. (canceled)

22. (canceled)

23. A sunscreen composition comprising:
(a) about 5 wt. % to about 50 wt. % of capsules of claim 1; and
(b) a cosmetically acceptable carrier.

24. A sunscreen composition comprising:
(a) about 5 wt. % to about 50 wt. %, based on the total weight of the sunscreen composition, of photosensitive capsules having an average diameter size of about 50 nm to about 850 μm, wherein the capsules comprise;
(i) about 10 wt. % to about 60 wt. %, based on the total weight of the capsules, of a polymeric photosensitive degradable shell comprising a polymer having photo-responsive groups that react in response to UV light to degrade the polymer,
wherein the polymer is selected from the group consisting of a polyacrylate, a polyurea, a polyurethane, polyester, cellulose, starch, a polysaccharide, and a silicone, and
wherein the photo-responsive groups are selected from the group consisting of an azobenzene group, a nitrobenzyl group, a quinone-methide group, and an o-nitrobenzyl group; and
(ii) about 10 wt. % to about 75 wt. %, based on the total weight of the capsules, of one or more UV filters encapsulated by the photosensitive degradable shell; and
(b) a cosmetically acceptable carrier.

25. A sunscreen composition according to claim 24 that is transparent.

26. A method of protecting a keratinous substrate from UV radiation comprising applying a sunscreen composition of claim 1 to the keratinous substrate.

27. (canceled)
